(19) United States
(12) Patent Application Publication
(10) Pub. No.: US 2005/0159773 A1
(43) Pub. Date: Jul. 21, 2005

(54) Expandable retrieval device with dilator tip

(75) Inventors: Thomas E. Broome, Shakopee, MN (US); Anthony C. Vrba, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/762,683

(22) Filed: Jan. 20, 2004

(51) Int. Cl. A61M 29/00

(52) U.S. Cl. 606/200; 606/198

(57) ABSTRACT

Devices and systems for retrieving an intravascular device within a body lumen are disclosed. The retrieval device may comprise an elongated tubular member having a relatively stiff proximal segment, an expandable distal segment, and an inner lumen disposed about a guidewire. The distal segment may include a braid configured to expand when axially compressed. A dilator tip operatively coupled to the elongated tubular member and guidewire may be used to track the retrieval device through the body.

### FIELD OF THE INVENTION

The present invention relates generally to the field of medical devices. More specifically, the present invention pertains to devices and systems for retrieving intravascular devices.

### BACKGROUND OF THE INVENTION

Intravascular devices such as an embolic protection filters are typically placed in a vessel such as an artery or vein to filter emboli contained in the blood stream. Examples of procedures employing such filters include angioplasty, atherectomy, thrombectomy, and stenting. These procedures generally involve transluminally inserting and delivering within an artery or vein an elongated wire and filter to a location distal a lesion. Once placed, a therapeutic device such as an angioplasty catheter is advanced along the wire to the site of the lesion to perform a therapeutic procedure (e.g. percutaneous transluminal coronary angioplasty). A stent can also be advanced to the site of the lesion and engaged along the wall of the vessel to prevent restenosis from occurring within the vessel.

Retrieval of the embolic protection filter generally involves the use of a catheter or sheath having an inner lumen configured to collapse the filter and captured emboli therein. The ability of such retrieval devices to effectively trap the filter and its contents may depend in part on the size of the filter and guidewire, the profile of the sheath, and the amount of emboli collected. Other factors such as the complexity of the sheath may also affect the ability of the retrieval sheath to capture the filter. Current retrieval systems are either too complicated due to the necessity of an actuating mechanism to capture the filter, or are difficult to track through the vasculature due to the shape of the sheath.

### SUMMARY OF THE INVENTION

The present invention pertains to devices and systems for retrieving intravascular devices. A retrieval device in accordance with an exemplary embodiment of the present invention may include an elongated tubular member having a proximal segment, a distal segment, and an inner lumen disposed at least in part therethrough. The proximal segment may comprise a relatively stiff and rigid material that allows the user to manipulate the retrieval device within the body. The distal segment may comprise an elastic material adapted to radially expand to encompass an intravascular device therein.

In certain embodiments, a braided layer coupled to or formed integrally with the distal segment may be utilized to impart expandability to the distal segment. The braided layer may comprise a number of filaments encased along all or a portion of the distal segment. Factors such as the material composition, shape, or thickness of the filaments can be selected to impart a particular characteristic to the distal segment such as expandability or radiopacity.

The retrieval device may further include a dilator tip that can be used to facilitate tracking of the retrieval device along a guidewire. The dilator tip may include a proximal segment having a size and shape that can be tightly fit within the distal segment. The distal section of the dilator tip may have a generally conical shape that tapers in the distal direction. In use, the relatively small profile at the distal end of the dilator tip provides a gradual transition that reduces trauma to the body, and prevents interference from occurring as the retrieval device and tip are advanced along the guidewire beyond other intravascular devices (e.g. a stent). In addition, the dilator tip maintains the retrieval device in a centered position along the guidewire, further reducing interference and/or trauma within the body.

### BRIEF DESCRIPTION OF THE DRAWINGS

FIG. 1 is a partial cross-sectional view of a retrieval device in accordance with ail exemplary embodiment of the present invention;

FIG. 2 is a partial cross-sectional view of the retrieval device of FIG. 1, showing the showing the distal segment in an unexpanded state prior to insertion of the dilator tip;

FIG. 3 is a plan view of an embolic protection filter disposed within a vessel distal a lesion and placed stent;

FIG. 4 is a plan view of the vessel shown in FIG. 3, wherein a retrieval device is shown advanced along the guidewire across the stent and engaged against the stop;

FIG. 5 is a plan view of the vessel shown in FIG. 3, wherein the retrieval device is shown further advanced along the guidewire in order to collapse the embolic protection filter; and

FIG. 6 is a plan view of the vessel shown in FIG. 3, wherein the embolic protection filter is shown collapsible within the retrieval device.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

FIG. 1 is a perspective view of a retrieval device 10 in accordance with an exemplary embodiment of the present invention. Retrieval device 10 comprises an elongated tubular member 12 having a proximal segment 14, a distal segment 16, and an inner lumen 18 disposed through at least part of the elongated tubular member 12. The inner lumen 18 can be dimensioned to slidably receive a guidewire 20 or other suitable guiding member.

The proximal segment 14 may be formed from a suitable stiff material having sufficient column strength and rigidity to withstand buckling or bulging as the retrieval device 10 is advanced over the guidewire 20 and engaged about an intravascular device. The wall thickness of the proximal segment 14 may be generally uniform along the length of the retrieval device 10, or may vary to alter the stiffness or torqueability characteristics of the device 10, as desired. In the embodiment of FIG. 1, for example, the proximal segment 14 may decrease in thickness from the proximal end of the retrieval device 10 (not shown) towards the distal end 22 of the proximal segment 14, resulting in a gradual reduction in stiffness along the length of the proximal segment 14. In other embodiments, the proximal segment 14 may have a constant thickness along its length to provide a uniform stiffness along the segment 14, if desired.

The proximal segment 14 may be formed at least in part from a polymeric material such as polyether block amide (PEBA), which is commercially available from Atochem Polymers of Birdsboro, Pennsylvania under the trade name PEBAX. Other suitable polymeric materials frequently used in the construction of catheters shafts or retrieval sheaths may be employed. The proximal segment 14 may comprise one or more segments having differing material characteristics such as stiffness, torsional rigidity, tensile strength, and/or hardness. In some embodiments, the material(s) used to form the proximal segment 14 may differ from the material(s) used to form the distal segment 16 to impart a particular characteristic to the retrieval device 10. For example, the material forming the proximal segment 14 may have a relatively low modulus of rigidity and elasticity than the material forming the distal segment 16, imparting greater stiffness and torqueability to the proximal segment 14. This increased stiffness and torsional rigidity facilitates the efficient transference of axial and rotational movement through the proximal segment 14 as the physician manipulates the retrieval device 10 within the body. The distal segment 16 comprising the less stiff and rigid material is thus capable of greater bending to permit the retrieval device 10 to be inserted into difficult to reach areas such as a branching vessel, for example.

The distal segment 16 may be configured to radially expand and encompass an intravascular device therein. The expandability of the distal segment 16 may be due at least in part to the selection of materials used to form the segment 16. Examples of materials that can be used in the construction of the distal segment 16 may include, but are not limited to, polyethylene terapthalate (PET), polytetrafluoroethylene (PTFE), polyurethane (Nylon) fluorinated ethylene propylene (FEP), ethylene tetrafluoroethylene (ETFE), polyurethane, polypropylene (PP), polyvinylchloride (PVC), polyether-ester, polyester, polyamide, elastomeric polyamides, block polyamide/ethers, polyether block amide (PEBA), silicones, polyethylene (PE), polyether-ether ketone (PEEK), polyimide (PI), polyetherimide (PEI), polyphenylene sulfide (PPS), polyphenylene oxide (PPO), polysulfone, perfluoro(propyl vinyl ether) (PFA), or other suitable materials, mixtures, combinations or copolymers thereof. In certain embodiments, the polymeric material may be blended with or otherwise include a liquid crystal polymer (LCP) to enhance torqueability.

The material forming the proximal segment 14 and/or distal segment 16 may include a radiopaque filler such as barium sulfate (BaSO₄) or bismuth subcarbonate ((BiO)₂CO₃) to permit visualization of the retrieval device 10 within the body. Radiopaque materials are understood to be materials capable of producing a relatively bright image on a fluoroscopic monitor or other imaging device. When a radiopaque die is injected into the vessel at issue, the relatively bright image produced on the monitor can be used to determine the location of the retrieval device 10 within the body.

A braided layer 24 coupled to or formed integrally with the distal segment 16 of the elongated tubular member 12 may be utilized to impart expandability to the distal segment 16 while maintaining the stiffness and rigidity characteristics of the retrieval device 10. The braided layer 24 may include a number of filaments 26 encased within or disposed adjacent to the distal segment 16. The filaments 26 may be arranged generally in two sets of parallel helices wound in opposite directions about a common longitudinal axis generally coincident with the guidewire 20. The filaments 26 may intersect each other in an overlapping or interwoven fashion to permit the distal segment 16 to radially expand when subjected to a compressive force. In the exemplary embodiment depicted in FIG. 1, the braided layer 24 extends along the entire length of the distal segment 16, terminating proximally at or near the distal end 22 of the proximal segment 14. In other embodiments (not shown), however, the braided layer 24 may extend along only a portion of the distal segment 16, or may extend further into all or a portion of the proximal segment 14.

The filaments 26 can be made from any number of suitable materials including polymers, metals, metal alloys, metal-polymer composites, or metal-metal composites. Some examples of suitable metals and metal alloys include platinum, stainless steel (e.g. 304 or 316 stainless), nickel-titanium alloy (Nitinol), nickel-chromium alloy, nickel-chromium alloy, cobalt alloy, or the like. Polymers similar to that used in the construction of the proximal and distal segments 14, 16 may also be used in forming the filaments 26. The filaments 26, or portions thereof, may also be doped with or otherwise include a radiopaque material to facilitate fluoroscopic visualization within the body. For example, the filaments 26 may be formed at least in part of gold, platinum, palladium, tantalum, tungsten alloy or other suitable material capable of producing a relatively bright image on a fluoroscopic screen or other imaging device.

In certain embodiments, the filaments 26 may be formed from a composite material configured to impart one or more desired characteristics to the braided layer 24. For example, one or more stainless steel and nickel-titanium alloy wires can be wound together to form filaments having a desired characteristic such as superelasticity. Alternatively, in those embodiments employing round wire or flat ribbon, for example, a composite material formed by a drawing, cladding or other suitable process may used to form filaments having a desired characteristic such as radiopacity.

Other characteristics such as the shape and thickness of the filaments 26 forming the braided layer 24 may also vary to alter the characteristics of the retrieval device 10. In the exemplary embodiment depicted in FIG. 1, the filaments 26 forming the braided layer 24 22 are made from monofilament wire having a generally round transverse cross-sectional area. Other filament configurations may be employed, however, such as flat ribbon, multi-filament wire, threads, fibers, or combinations thereof. The thickness of the filaments 26 may vary in dimension to impart a greater or lesser amount of resistance to radial expansion to the distal segment 16. In general, the larger the size of filaments employed, the greater the resistance to radial expansion that results.

The retrieval device 10 may further include a dilator tip 28 for improved tracking through the vasculature. Dilator tip 28 may include a proximal section 30, a distal section 32, and an inner lumen 34 disposed therethrough adapted to slidably receive the guidewire 20. The dilator tip 28 may have a generally circular transverse cross-sectional area that is configured to fit at least in part within the inner lumen 18 of the distal segment 16. The distal section 32 of the dilator tip 28 has a generally conical shape that tapers in the distal direction. In use, the relatively small profile at the distal end 36 of the dilator tip 28 provides a gradual transition that reduces trauma to the body, and prevents interference from occurring as the retrieval device and dilator tip 28 are advanced along the guidewire 20 beyond other intravascular devices. The dilator tip 28 further aids in maintaining the retrieval device 10 in a centered position along the guidewire 20, thereby improving the ability of the device 10 to cross stents or other placed intravascular devices, and to facilitate tracking through, for example, a tortuous or narrowed vessel. In certain embodiments, the dilator tip 28 may include a radiopaque material, marker band or other visualization means, allowing the user to fluoroscopically monitor the location of the dilator tip 28 within the body.

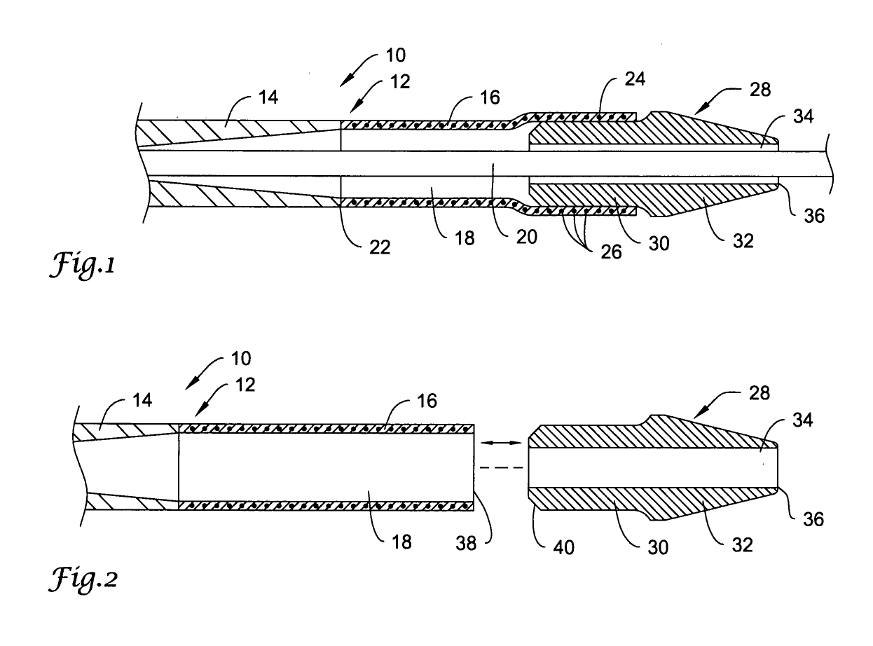

FIG. 2 is a partial cross-sectional view of the retrieval device 10 of FIG. 1, showing the distal segment 16 in an unexpanded state prior to insertion of the dilator tip 28. As shown in FIG. 2, the distal segment 16 may have a substantially uniform profile along its length with an inner diameter slightly smaller than the outer diameter of the dilator tip 28. The relative dimensions of the dilator tip 28 and distal segment 16 can be selected to provide an interference fit between the two members, allowing the dilator tip 28 to tightly fit within the distal segment 16. In use, this interference fit maintains the dilator tip 28 in a fixed position relative to the distal segment 16 as the retrieval device 10 is advanced through the body.

To insert the dilator tip 28 into the distal segment 16, the proximal section 30 of dilator tip 28 is inserted into the opening 38 at the distal end of the retrieval device 10 and compressed therein, as indicated by the arrow in FIG. 2. A taper 40 on the proximal end of the dilator tip 28 may be used to guide the dilator tip 28 as it is initially compressed into the inner lumen 18. Compression of the dilator tip 28 into the distal segment 16 causes the segment 16 to expand about the proximal section 30 of the dilator tip 28 to a position similar to that depicted in FIG. 1. The dilator tip 28 can be subsequently withdrawn from within the inner lumen 18, if desired, causing the distal segment 16 to revert to its initial (i.e. unexpanded) state.

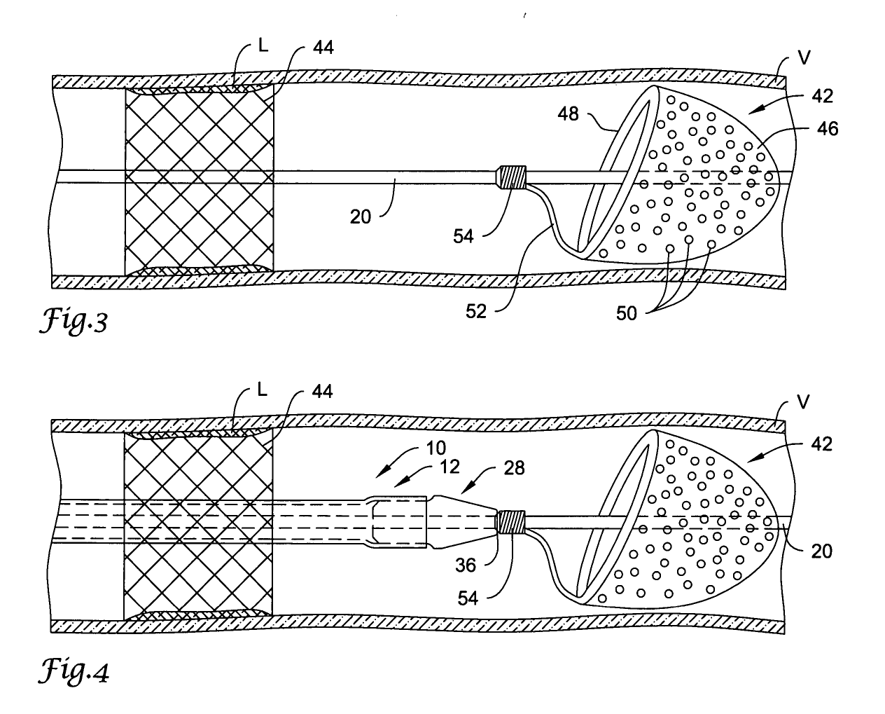

Referring now to FIGS. 3-6, an exemplary method of retrieving an intravascular device in accordance with the present invention will now be discussed with respect to retrieval device 10 described herein. In a first position depicted in FIG. 3, an illustrative embolic protection filter 42 is shown coupled to a guidewire 20 positioned within a blood vessel V distal a lesion L. A previously placed stent 44 is also shown advanced along the guidewire 20 and positioned across the site of the lesion L to prevent restenosis from occurring subsequent to a therapeutic procedure such as an angioplasty or atherectomy.

Embolic protection filter 42 may include a filter membrane 46 operatively coupled to a support hoop 48 that supports the filter membrane 46 in an expanded position within the vessel V. The support hoop 48 can be configured to self-expand when unconstrained radially, biasing the filter membrane 46 to expand within the vessel V. The filter membrane 46 may be made from a biocompatible polymeric material having a number of openings or apertures 50 configured to collect embolic debris disposed in the vessel V without significantly impeding the flow of blood. All or portions of the embolic protection filter 42 can be coated with an anti-thrombogenic coating such as Heparin or its equivalent to discourage clot formation on the filter 42.

The support hoop 48 may be connected to the guidewire 20 via one or more struts 52 extending proximally from the support hoop 48 to a stop 54. Stop 54 can include a clamp or wire winding, solder or other suitable connector coupling the proximal portion of the filter 42 to the guidewire 20. The portion of the filter membrane 46 located at or near the distal end of the embolic protection filter 42, in turn, can be attached to the guidewire 20 by, for example, an adhesive process.

To retrieve the embolic protection filter 42 from the vessel V, the physician inserts the dilator tip 28 into distal segment 16 of the elongated tubular member 12, as described previously with respect to FIG. 2. With the dilator tip 28 inserted into the distal segment 16, the physician next inserts the proximal end of the guidewire 20 into the distal end 36 of the dilator tip 28 and threads the guidewire 20 through the inner lumen 34 and 18. The physician then inserts the retrieval device 10 and attached dilator tip 28 into the vasculature via a small puncture wound formed, for example, in the femoral or jugular veins, and advances the device 10 and dilator tip 28 to a target location within a vessel. The retrieval device 10 can be advanced via an other-the-wire approach, wherein the retrieval device 10 is advanced along a substantial part of the length of the guidewire 20. Alternatively, the retrieval device 10 can be advanced via a single operator exchange (SOE) approach, wherein an exit port located along the side of the elongated tubular member 12 can be used to advance only a portion of the retrieval device 10 along the guidewire 20.

FIG. 4 is a plan view showing the retrieval device 10 advanced along the guidewire 20 across the site of the stent 44 and engaged against the stop 54. As shown in FIG. 4, the dilator tip 28 maintains the retrieval device 10 in a central position about the guidewire 20, reducing the likelihood that the device 10 will interfere with the stent 44 during insertion and/or removal.

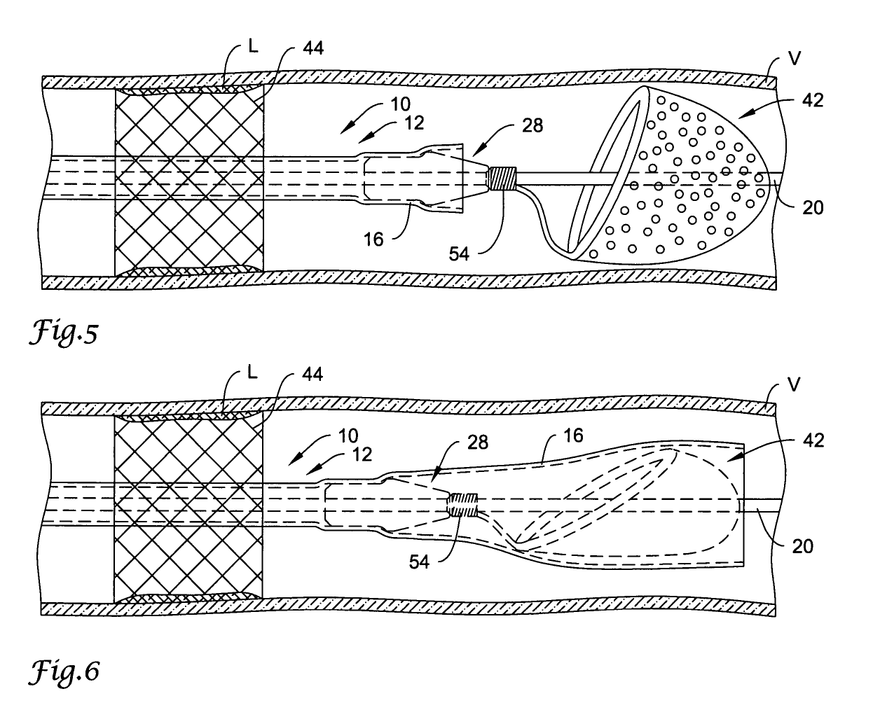

The distal end 36 of the dilator tip 28 is configured to engage the stop 54, which is prevents further movement of the dilator tip 28 in the distal direction along the guidewire 20. With the dilator tip 28 engaged against the stop 54, the physician next advances the elongated tubular member 12 distally while holding the guidewire 20 stationary, causing the initiation of the radial expansion of distal segment 16 and subsequent advancement distally over the dilator tip 28, as shown in FIG. 5. The shape of the dilator tip 28 causes the elongated tubular member 12 to flare outwardly as it is advanced distally. Continued movement of the elongated tubular member 12 in the distal direction causes the distal segment 16 to further expand radially and encompass the embolic protection filter 42, causing the filter 42 to collapse completely therein, as shown in FIG. 6. The retrieval device 10, embolic protection filter 42, and guidewire 20 can then be removed from the vessel V.

While FIGS. 3-6 specifically illustrate the removal of an embolic protection filter from the body, it is contemplated that any number of other intravascular devices may be retrieved and/or delivered with the present invention. Examples of other intravascular devices may include stents, clot pullers, vena cava filters, atherectomy devices, angioplasty devices, or the like.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

1. A medical device, comprising:
an elongated tubular member having a proximal segment, a distal segment, and an inner lumen disposed at least partially therethrough, the distal segment configured to radially expand when axially compressed; and
a dilator tip insertable at least in part within the distal segment.

2. The medical device of claim 1, wherein the proximal segment varies in thickness along its length.

3. The medical device of claim 1, wherein the distal segment includes a braid.

4. The medical device of claim 1, wherein the dilator tip has a generally circular transverse cross-sectional area.

5. The medical device of claim 1, wherein the dilator tip has a proximal section, a distal section, and an inner lumen disposed therethrough.

6. The medical device of claim 5, wherein the proximal section of said dilator tip is configured to tightly fit within the distal segment.

7. The medical device of claim 5, wherein the distal section of said dilator tip is distally tapered.

8. The medical device of claim 1, wherein the elongated tubular member is configured to radially expand and encompass an intravascular device therein.

9. The medical device of claim 8, wherein the intravascular device is an embolic protection filter.

10. The medical device of claim 1, wherein the elongated tubular member is configured for use over-the-wire.

11. The medical device of claim 1, wherein the elongated tubular member is configured for single operator exchange.

12. A medical device, comprising:
an elongated tubular member having a proximal segment, a distal segment, and an inner lumen disposed at least partially therethrough, the distal segment including a braid configured to radially expand when axially compressed; and
a dilator tip insertable at least in part within the distal segment.

13. The medical device of claim 12, wherein the proximal segment varies in thickness along its length.

14. The medical device of claim 12, wherein the dilator tip has a generally circular transverse cross-sectional area.

15. The medical device of claim 12, wherein the dilator tip has a proximal section, a distal section, and an inner lumen disposed therethrough.

16. The medical device of claim 15, wherein the proximal section of said dilator tip is configured to tightly fit within the distal segment.

17. The medical device of claim 15, wherein the distal section of said dilator tip is distally tapered.

18. The medical device of claim 12, wherein the elongated tubular member is configured to radially expand and encompass an intravascular device therein.

19. The medical device of claim 18, wherein the intravascular device is an embolic protection filter.

20. The medical device of claim 12, wherein the elongated tubular member is configured for use over-the-wire.

21. The medical device of claim 12, wherein the elongated tubular member is configured for single operator exchange.

22. A medical device, comprising:
an elongated tubular member having a proximal segment, a distal segment, and an inner lumen disposed at least partially therethrough, the distal segment configured to radially expand when axially compressed; and
a dilator tip insertable at least in part within the distal segment, the dilator tip having a proximal section configured to tightly fit within the distal segment, a distal section, and an inner lumen disposed therethrough.

23. The medical device of claim 22, wherein the proximal segment varies in thickness along its length.

24. The medical device of claim 22, wherein the distal segment includes a braid.

25. The medical device of claim 22, wherein the dilator tip has a generally circular transverse cross-sectional area.

26. The medical device of claim 22, wherein the distal section of said dilator tip is distally tapered.

27. The medical device of claim 22, wherein the elongated tubular member is configured to radially expand and encompass an intravascular device therein.

28. The medical device of claim 27, wherein the intravascular device is an embolic protection filter.

29. The medical device of claim 22, wherein the elongated tubular member is configured for use over-the-wire.

30. The medical device of claim 22, wherein the elongated tubular member is configured for single operator exchange.

31. A system for retrieving an intravascular device disposed within a body lumen, comprising:
an embolic protection filter disposed about an elongated wire;
a retrieval device configured to radially expand and encompass the intravascular filter therein, said retrieval device comprising an elongated tubular member having a proximal segment, a distal segment, and an inner lumen adapted to slidably receive the elongated wire; and
a dilator tip insertable at least in part within the distal segment, said dilator tip configured to engage a stop disposed about the elongated wire.

32. A system for retrieving an intravascular device disposed within a body lumen, comprising:
an embolic protection filter disposed about an elongated wire;
a retrieval device configured to radially expand an encompass the intravascular filter therein, said retrieval device comprising an elongated tubular member having a proximal segment, a distal segment, and an inner lumen adapted to slidably receive the elongated wire; and
a dilator tip insertable at least in part within the distal segment, the dilator tip including a proximal section configured to tightly fit within the distal segment, a distal section configured to engage a stop disposed about the elongated wire, and an inner lumen disposed therethrough configured to slidably receive the elongated wire.
